(19) United States
(12) Patent Application Publication
(10) Pub. No.: US 2002/0019421 A1
(43) Pub. Date: Feb. 14, 2002

(54) Compositions and therapy for substance addiction

(76) Inventor: Roni Biberman, Tel-Aviv (IL)

Correspondence Address: Eitan, Pearl, Latzer & Cohen-Zedek, One Crystal Park, Suite 210, 2011 Crystal Drive, Arlington, VA, 22202-3700, US

(21) Appl. No.: 09/898,027

(22) Filed: Jul. 5, 2001

Related U.S. Application Data
(60) Provisional application No. 60/216,366, filed on Jul. 5, 2000.

(51) Int. Cl. A61K 31/44; A61K 9/00

(52) U.S. Cl. 514/343; 424/43

(57) ABSTRACT

Inhibitors of monoamine oxidase used in combination with an addictive substance, or a pharmacological derivative or analogue thereof, are useful for the treatment of substance addiction disorders. In particular, the invention discloses compositions, and methods of use thereof, comprising selegiline and nicotine for the treatment of cigarette smokers wishing to abstain. The compositions and methods of use thereof include oral, inhalant, parenteral and transdermal patch modes of therapy, whereby the subject benefits from the combined effects of a monoamine oxidase inhibitor in combination with an addictive substance, or derivative thereof.

### FIELD OF THE INVENTION

The present invention relates to medicinal compositions and methods of treating substance addiction, in particular addiction to nicotine-containing tobacco products, wherein administration to an addicted subject of inhibitors of monoamine oxidase combined with co-administration of an addictive substance is used to alleviate withdrawal symptoms and to change or eliminate patterns of addictive substance consumption, in particular that due to cigarette smoking, over both short and extended periods of time.

### BACKGROUND OF THE INVENTION

The present disclosure is related to U.S. provisional patent application No. 60/216,366, filed on Jul. 5, 2000 and which is hereby is incorporated herein by reference. The statistical risk of dying from lung cancer in the United States has doubled in the past thirty years for male cigarette smokers and has quadrupled for female cigarette smokers. Lung cancer has now displaced cardiovascular disease as the single most important cause of excess mortality among smokers. Yet, about 50 million Americans continue to smoke.

The benefits for smoking cessation are many, and are summarized in a publication entitled “The Health Benefits of Smoking Cessation: A Report of the Surgeon General, 1990”, available from the Office on Smoking and Health, Center for Disease Control, Rockville, Md. Among the benefits summarized are that within twenty-four hours the chance of a heart attack decreases, within about two weeks to three months lung function increases up to thirty percent, and in one year the excess risk of coronary heart disease becomes half that of a smoker.

Smoking cessation programs often address both the physiological factor and the psychological factor. The widely available “nicotine-releasing patches” have been highly advertised, and are useful for maintaining a nicotine-habituated patient on nicotine while addressing further the psychological factors of addiction. Craving is the most consistently observed withdrawal symptom and is probably best described as a combination of physiological and psychological factors. To treat craving for nicotine, other modes of nicotine replacement therapy have been introduced, including administration of nicotine by oral chewing gum and by inhalation with a carrier for absorption through the respiratory mucosa, including the lungs.

In addition to nicotine replacement therapy, other pharmacological therapies have been described based on compounds which are expected to influence the physiological mechanisms common to nicotinic agents. This is the basis for the use of compounds which interact with acetylcholine receptors, or affect levels of the neurotransmitter acetylcholine, as a means for reducing the desire for nicotine or other addictive drugs. Atropine and scopolamine are alkaloids that block the action of acetylcholine at muscarinic receptors to produce antispasmodic, antisecretory, and antiparkinsonism actions. Physostigmine is an inhibitor of the enzyme acetylcholinesterase, thereby raising the level of acetylcholine through inhibition of its metabolism.

The acetylcholine receptors appear to be of two general subtypes, One subtype appears to have nicotine as an agonist or muscarine as an agonist. Both types of receptor are found in the central nervous system as well as the autonomic nervous system.

Other compounds in addition to cholinergic and anticholinergic drugs have been tested for the purpose of attenuating withdrawal symptoms, including craving. These include benzodiazepines, dopamine agonists, serotonin agonists, inhibitors of dopamine carboxylase, and inhibitors of serotonin reuptake or of dopamine reuptake. The neurotransmitter dopamine has been shown to be strongly linked to the internal mechanism of pleasure sensation mechanism of animal, including human, brains.

More recently developed strategies have focused on the genetic basis of drug addiction, in particular addiction to nicotine. Thus it has been hypothesized that individuals in whom nicotine is slowly metabolized are both more likely to consume fewer cigarettes or not be smokers at all. Thus inhibition of the metabolism of nicotine, through either inhibition of the nicotine degrading enzymes or through inhibition of the synthesis of nicotine degrading enzymes.

Inhibitors of the intracellular enzyme monoamine oxidase (monoamine oxidase) have been traditionally used in medical therapies for the treatment of disease of the central nervous system, in particular depression and Parkinson's disease. The use of monoamine oxidase inhibitors in the treatment of addiction, in particular, tobacco product addiction, has been recurrently suggested although, for lack of conclusive data, these therapeutic compounds are not currently endorsed for this use by the medical establishment.

Most significantly, despite the introduction of nicotine replacement therapies, most frequently administered in the form of transdermal patch or nicotine gum, addiction to tobacco products and the associated increased morbidity and mortality continues to be a global epidemic.

### SUMMARY OF THE INVENTION

The invention provides a pharmaceutical composition comprising an amount of an inhibitor of monoamine oxidase, an amount of an addictive substance, or derivative thereof, and a suitable diluent or carrier.

The invention provides a pharmaceutical composition comprising an amount of an inhibitor of monoamine oxidase, an amount of an addictive substance, or derivative thereof, and a suitable diluent or carrier, wherein said composition is adapted to administer both the inhibitor of monoamine oxidase and the addictive substance, or derivative thereof, via the same route of administration.

The invention provides a pharmaceutical composition comprising an amount of an inhibitor of monoamine oxidase, an amount of an addictive substance or derivative thereof, and a suitable diluent or carrier, wherein said carrier is an oral tablet, capsule, a comestible article, an aerosol, nebulized vapor, smoke, or a transdermal patch, and wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration.

The invention provides a pharmaceutical composition comprising an amount of an inhibitor of monoamine oxidase, an amount of an addictive substance or derivative thereof, and a suitable diluent or carrier, wherein said addictive substance, or derivative thereof, is nicotine and said carrier is an oral tablet, capsule, a comestible article, an aerosol carrier a vapor, a smoke, or a transdermal patch and wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration,

The invention provides a pharmaceutical composition comprising an amount of an inhibitor of monoamine oxidase, an amount of an addictive substance or derivative thereof, and a suitable diluent or carrier, wherein said i monoamine oxidase in inhibitor is selegiline and said carrier is an oral tablet, capsule, a comestible article, an aerosol, a vapor, a smoke, or a transdermal patch, and wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration.

The invention provides a pharmaceutical composition comprising an amount of an inhibitor of monoamine oxidase, an amount of an addictive substance, or derivative thereof, and a suitable diluent or carrier, wherein said monoamine oxidase inhibitor is selegiline, said addictive substance, or derivative thereof, is nicotine, and said carrier is an oral tablet, capsule, a comestible article, an aerosol, a vapor, a smoke, or a transdermal patch, and wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration.

The invention provides a pharmaceutical composition comprising an amount of an inhibitor of monoamine oxidase, an amount of an addictive substance or derivative thereof, and a suitable diluent or carrier, wherein said addictive substance, or derivative thereof, is nicotine, said carrier is an oral tablet or capsule, a comestible article, an aerosol, a vapor, a smoke, or a transdermal patch, wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration, and wherein said inhibitor of monoamine oxidase is selected from the group consisting of selegiline, desmethylselegiline, S(+)desmethylselegiline, lazabemide, broformine, rasagiline, LU-53439, LU-43839, idazoxan, clorgyline; cimoxatone, cirazoline; befloxatone; brofaromine; bazinaprine; MD-240928; BW-616U; BW-1370U87; CS-722; E-2011; harmine; harmane, norharmane; harmaline; moclobemide; PharmaProjects 3975 ; RO 41-1049; RS-8359; T-794; toloxatone; K-Y 1349; LY-51641; LY-121768; M&B 9303; MDL 72394; MDL 72392; sercloremine; MD 72394; MO 1671, amiflamine; vanoxerine; AGN 2253, iproniazid; isocarboxazid; M-3-PPC; nialamid; phenelzine; pargyline; tranylcypromine, budipine; caroxazone; D-1711; fezolamine; FLA-334; FLA-289; K-11566; K-11829, metralindole; MPCPAM; PharmaProjects 227; PharmaProjects 2806; PharmaProjects 1122; PharmaProjects 331; PharmaProjects 4433; RS-2232; UP-614-04, or mixtures thereof.

The invention provides a pharmaceutical composition, wherein said monoamine oxidase inhibitor is selegiline, said carrier is an oral tablet, a capsule, a comestible article, an aerosol, a vapor, a smoke, or a transdermal patch, and wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration, and said addictive substance, or derivative thereof, is nicotine, cotinine, a nicotinic agonist, a derivative of nicotine, a dopamine agonist, an opiate, morphine, heroin, methadone, buprenorphine, fentanyl, naloxone, codeine, an amphetamine, metamphetamine, methylphenidate, methylenedioxymethamphetamine, a dopamine re-uptake inhibitor, an alkaloid, cocaine, or derivative thereof. The invention provides a pharmaceutical composition, wherein said monoamine oxidase inhibitor is selegiline, wherein said addictive substance, or derivative thereof, is nicotine, said carrier is an oral tablet, a capsule, a comestible article, an aerosol, a vapor, a smoke, or a transdermal patch, wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration, and said composition further comprises at least one compound selected from the group consisting of: an inhibitor of nicotine metabolism; a nicotine antagonist; a nicotine agonist; an inhibitor of CYP2A6, a beta-blocker; an alpha-2 adrenergic anatagonist; a muscle relaxant; a benzodiazepine; an inhibitor of nicotine metabolism; a nicotine antagonist; a peripheral nicotine anatagonist; a nicotine agonist, a dopamine agonist; a dopamine antagonist; a serotonin agonist; a serotonin antagonist; an antihistamine; a cannabinoid receptor antagonist; a cannabinoid receptor agonist; an anticholinergic agent; an inhibitor of acetylcholine esterase; a muscarinic antagonist; a muscarinic agonist; an opiate agonist; an opiate anatogonist; an amphetamine agonist; an amphetamine antagonist; a glutamate antagonist; a NMDA antagonist; an antihypertensive; an inhibitor of serotonin transport (re-uptake); an inhibitor of dopamine transport (re-uptake); an inhibitior of noradrenaline transport (re-uptake); a NaSSA; an NMDA antagonist; an analgesic; a vasodilator; a calcium channel antagonist; a xanthine; an inhibitor of COMT (catechol-O-methyltransferase); a steroid; a steroid hormone; a vitamin; an inhibitor or monoamine oxidase; an antioxidant; an atypcial antidepressant; an atypical antipsychotic; an addictive substance; a pharmacological analogue of an addictive substance; or mixtures thereof.

The invention provides a method of treating a subject addicted to an addictive substance or product comprising the steps of co-administering to the subject in need thereof a pharmaceutical composition comprising a therapeutically effective amount of selegiline and a pharmaceutical composition comprising a therapeutically effective amount of nicotine, thereby attenuating consumption of said substance or product.

The invention provides a method of treating a subject addicted to an addictive substance or product comprising the steps of co-administering to the subject in need thereof a pharmaceutical composition comprising a therapeutically effective amount of selegiline in oral tablet, capsule, comestible dosage, aerosol, vapor, smoke, or transdermal patch dosage form, wherein said dosage form is suitable for parenteral administration, including intradermal, subcutaneous, intramuscular, intravenous, intrathecal, sublingual, rectal, vaginal, intraocular, transdermal, respiratory mucosal, or pulmonary routes of administration, and a pharmaceutical composition comprising a therapeutically effective amount of nicotine in a transdermal patch dosage form, thereby attenuating consumption of said substance or product.

The invention provides a method of treating a subject addicted to an addictive substance or product comprising the steps of administering to the subject in need thereof a pharmaceutical composition comprising a therapeutically effective amount of selegiline and a therapeutically effective amount of nicotine combined in an oral tablet, capsule, comestible, aerosol, vapor, smoke, or transdermal dosage form, wherein said dosage form is suitable for parenteral administration, including intradermal, subcutaneous, intramuscular, intravenous, intrathecal, sublingual, rectal, vaginal, intraocular, transdermal, respiratory mucosal, or pulmonary routes of administration, thereby attenuating consumption of said substance or product.

The invention provides a method of treating the symptoms of nicotine withdrawal comprising the steps of co-administering to the subject in need thereof a pharmaceutical composition comprising a therapeutically effective amount of selegiline, combined with a suitable diluent or carrier, and a pharmaceutical composition comprising a therapeutically effective amount of nicotine, combined with a suitable diluent or carrier, thereby alleviating the symptoms of nicotine withdrawal.

The invention provides a method of treating the symptoms of nicotine withdrawal comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in an transdermal patch dosage form, thereby alleviating the symptoms of nicotine withdrawal.

The invention provides a method of treating the symptoms of nicotine withdrawal comprising the steps of co-administering to the subject in need thereof a pharmaceutical composition a therapeutically effective amount of selegiline in oral tablet, capsule, or comestible dosage form and a pharmaceutical composition comprising a therapeutically effective amount of nicotine in an oral tablet, capsule, comestible, aerosol, vapor, smoke, or transdermal dosage form, thereby alleviating the symptoms of nicotine withdrawal.

The invention provides a method of treating the symptoms of nicotine withdrawal comprising the steps of administering to the subject in need thereof a pharmaceutical composition comprising a therapeutically effective amount of selegiline and a therapeutically effective amount of nicotine combined in an oral tablet, capsule, or comestible dosage form, thereby alleviating the symptoms of nicotine withdrawal.

The invention provides a method of treating the symptoms of nicotine withdrawal comprising the steps of administering to the subject in need thereof a pharmaceutical composition comprising a therapeutically effective amount of selegiline and a therapeutically effective amount of nicotine combined in a transdermal patch dosage form, thereby alleviating the symptoms of nicotine withdrawal.

The invention provides a method of treating a subject addicted to a tobacco product comprising the steps of co-administering to the subject in need thereof a pharmaceutical composition comprising a therapeutically effective amount of selegiline and a pharmaceutical composition comprising a therapeutically effective amount of nicotine, thereby attenuating tobacco consumption.

The invention provides a method of treating a subject addicted to a tobacco product comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in a transdermal patch dosage form, thereby attenuating tobacco consumption.

The invention provides a method of treating a subject addicted to a tobacco product comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in an oral tablet, capsule, or comestible dosage form, thereby attenuating tobacco consumption.

The invention provides a method of treating a subject addicted to a tobacco product comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in an oral tablet, capsule, comestible, aerosol, vapor, smoke, or transdermal dosage for thereby attenuating tobacco consumption.

The invention provides a method of treating a subject addicted to a tobacco product comprising the steps of co-administering to the subject in need thereof a pharmaceutical composition comprising a therapeutically effective amount of selegiline in a transdermal patch dosage form and a pharmaceutical composition comprising a therapeutically effective amount of nicotine in a transdermal patch dosage form, thereby attenuating tobacco consumption.

The invention provides a method of treating a subject addicted to a tobacco product comprising the steps of administering to the subject in need thereof a pharmaceutical composition comprising a therapeutically effective amount of selegiline and a therapeutically effective amount of nicotine combined in an oral tablet or comestible dosage form, thereby attenuating tobacco consumption.

The invention provides a method of treating a subject addicted to a tobacco product comprising the steps of administering to the subject in need thereof a pharmaceutical composition comprising a therapeutically effective amount of selegiline and a therapeutically effective amount of nicotine combined in a transdermal patch dosage form, thereby attenuating tobacco consumption.

The invention provides a method of treating a subject addicted to an addictive substance or product comprising the steps of administering to the subject in need thereof a pharmaceutical composition of the invention, thereby attenuating chronic craving symptoms of a subject addicted to said substance or product.

The invention provides a method of treating the symptoms of nicotine withdrawal comprising the steps of administering to the subject in need thereof a pharmaceutical composition of the invention, thereby alleviating the chronic craving symptoms of a subject addicted to nicotine.

The invention provides a method of treating a subject addicted to tobacco product consumption comprising the steps of administering to the subject in need thereof a pharmaceutical composition of the invention, thereby attenuating chronic tobacco consumption.

### DETAILED DESCRIPTION OF THE INVENTION

In one aspect of the present invention, a method for relieving craving in a nicotine-habituated person who is abstaining from or reducing nicotine intake is provided by administering at least two components individually or as an admixture of first and second components. The first component is an inhibitor of monoamine oxidase and the second component is an addictive substance or a non-addicitive derivative thereof. Co-administration of the two components or administration of a balanced admixture of the first and second components relieves craving, yet surprisingly results in substantially no undesirable physiological changes. Instead, practice of the invention appears to reduce not only craving for nicotine, or nicotine containing tobacco products, in persons who are reducing or abstaining from nicotine intake, but appears as well to reduce other cravings associated with withdrawal syndromes, such as weight gain. The invention is thus useful for treating a wide range of withdrawal syndromes, including those encountered in subjects addicted to opiates, amphetamines, or cocaine. The present invention is related to U.S. provisional patent application No. 60/216,366, filed on Jul. 5, 2000 and which is hereby incorporated herein by reference.

A particularly preferred composition of the invention takes the form of a tablet, a capsule, or a comestible article for oral administration or of a patch for transdermal administration wherein the first component is an inhibitor of monoamine oxidase and the second component is an addictive substance, such as nicotine, or pharmacological analogue thereof. The combination of the first and second components is useful and effective because the first class of drug (specific or non-specific inhibitor of monoamine oxidase) should influence the response to nicotine, or a similarly addictive substance, through affects on neurotransmitter pathways or metabolism. As demonstrated herein, the inhibitor of monoamine oxidase selegiline is effective in itself to reduce the desire or craving for an addictive substance, namely nicotine, Furthermore, administration of the inventive compositions comprising both classes of compounds or alternatively, co-administration of both classes of compounds by different vehicles, have been shown to lead to a reduced desire for nicotine, which is greater than when subjects are administered a comparative mode of therapy using only nicotine or selegeline alone.

Drug addiction, in particular tobacco product addiction, is a public health epidemic of global proportions, accounting for millions of premature deaths annually. While treatment regimens such as nicotine replacement therapies remain popular, the current best accepted success rate for smoking cessation provided by administration of nicotine via transdermal patch is about 20%. Possibly higher rates may be obtained when respiratory routes of nicotine replacement therapy, i.e. whereby an aerosol, a vapor, a nebulized vapor, a particulate-containing vapor, or a smoke, are improved and implemented into clinical practice.

In addition to direct replacement therapies, other drugs have been used or suggested to be useful for the treatment of substance abuse and addiction, among them being the class of drugs known as monoamine oxidase inhibitors, including selegiline. Combination therapy comprised of administration of both nicotine and selegiline together, by either identical or separate routes of administration, is disclosed herein as a highly effective method for the treatment of tobacco addiction, exhibiting a synergistic effect in comparison to that of administration of either agent alone and achieving cessation rates of greater than 40%.

The invention disclosed herein comprises the combination of a monoamine oxidase inhibitor, such as selegiline, with one or more modes of nicotine replacement therapy, for the treatment of addiction, in particular addiction to tobacco products. In particular the invention provides a pharamaceutical composition comprising an addictive substance, or a pharamceutical analogue or derivative thereof, in combination with an inhibitor of monoamine oxidase, for administration to the same subject as a pharmacological treatment, employed alone or as an adjunctive measure, to promote the process of withdrawal from habitual consumption of an addictive substance or product wherein an addictive substance is contained.

The terms addiction, substance addiction, substance abuse, or substance dependence are frequently used interchangeably. In general, the term substance addiction is used herein to refer to the chronic administration, usually self-administration, of a chemical compound, or class of chemical compounds, which is either itself known to exert noxious effects to health and/or the route of administration of the compound or product thereof creates substantial physiological insults, yet the subject afflicted with the addiction continues to crave, seek, or administer the substance despite the known risks to health. Such is the case with tobacco product addiction whereby the active substance, nicotine, is most typically administered via the act of inhalation of tobacco smoke, as in cigarette, cigar, or pipe smoking. Nicotine is known to be highly addictive, hence the efforts to aid the afflicted subject via administration of purified, concentrated nicotine in the form of a transdermal patch, buccoral, oral, or respiratory route which obviate the need to inhale noxious tobacco smoke for acquiring the desired dose of nicotine. Thus, a subject addicted to cigarette smoking, or another tobacco product, may make use of the invention while attenuating or completely ceasing consumption of the offending tobacco product, and yet avoid the withdrawal signs and symptoms associated with this action, especially craving, thus allowing the addicted subject to avoid complete or partial relapse, whereby the addicted subject reinstitutes the addictive behaviors associated with the addictive substance, such as cigarette smoking.

The term substance dependence usually is taken to refer to the psychological and behavioral aspects of chronic ingestion of a substance known to have adverse effects on health or daily functioning. Substance dependence is thus one specific aspect of substance addiction.

The term substance abuse usually is taken to refer to periodic or chronic consumption of a substance, in particular a psychoactive substance, the consumption of which is continued by the subject despite the presence or knowledge of the associated harmful effects. Substance abuse is thus one specific aspect of substance addiction.

An addictive substance is any compound, combination of compounds, or product thereof, known to be addictive to an animal subject, especially human beings. An addictive substance is often consumed by the addicted subject in a repetitive, compulsive manner in order to obtain a mood-enhancing or mood-stimulating effect or to diminish or postpone the mood-related dysphoria and other psychological, emotional, and physical effects experienced by the addicted subject upon discontinuation or withdrawal from the ordinary schedule of consumption of the addictive substance. Craving, defined as the desire for the addictive substance and/or the desire to partake of the behavior patterns associated with consumption of the addictive substance, is a critical feature of addictive disorders. Examples of addictive substances are opiates, including heroin, morphine, codeine, and natural or synthetic derivatives thereof; opiate-like analgesic drugs, including fentanyl, buprenorphine, and natural or synthetic derivatives thereof; nicotine and natural or synthetic derivatives thereof; tobacco and tobacco-containing products thereof; cocaine and natural or synthetic derivatives thereof; amphetamines and natural or synthetic derivatives thereof; ethyl alcohol and ethanol-containing products; caffeine and caffeine-containing products; and high carbohydrate diets, especially the habitual consumption of sugar products and sweets.

For implementation, of the compositions and methods of use thereof provided by the invention, a non-addictive derivative or analogue may be used in place of, or in addition to, the addictive substance. As defined herein, a “derivative thereof” of an addictive substance is a substance which shares relevant pharmacological properties in common with a particular addictive substance, so that the “derivative thereof” may be considered to be either a pharmacological analogue, partial analogue, or antagonist of the addictive substance, but which is either less addictive or non-addictive in comparison to said addictive substance when tested in animals, including humans. Thus, for example, methadone or naloxone may be used in addition to or instead of morphine, heroin, or another highly addictive opiate.

Substance addiction is considered by many to be a life-long illness which persists long after the consumption of the addictive substance is ceased. Thus, for example, cigarette smokers, cocaine addicts, heroin addicts, alcoholics, and amphetamine addicts often experience one or more relapses while attempting to abstain. These relapses may occur following months or years of abstinence and are typically invoked by experts in the field as evidence to demonstrate the strong psychological aspects of substance addiction. Alternatively, or in addition, individual addicts may be genetically predisposed to the condition of substance addiction.

Both nicotine and tobacco smoke are considered to be dangerous to health. Nicotine has vasoconstrictive properties and probably accounts for the substantial increased risk for smokers to develop ischemic vascular disease, leading to heart disease, stroke, and peripheral vascular disease. Tobacco smoke contains a multitude of known carcinogens, and as well, raises blood carbon monoxide levels which impact negatively on hemoglobin function.

Acute and chronic opiate, amphetamine, cocaine and ethanol use are also known to produce cardiovascular disorders. Thus use of the invention is a means for mitigating the cardiovascular complications associated with substance addiction disorders through providing for withdrawal from noxious addictive substances and products thereof.

In addition to nicotine, other psychostimulant substances considered to be highly addicting are cocaine, heroin and other opiates, the amphetamines, cocaine, caffeine, and sweets.

The goal of therapy or treatment of substance addiction is to reduce the amount and/or rate of intake of the substance over time and as well, to reduce the rate of relapse. It is well known that individuals afflicted with an addictive condition who succeed in obtaining a reduction or complete cessation of intake of the substance to which they are addicted remain at a substantial risk to relapse during the course of their lifetime. To cure, or completely eradicate, an addictive condition over the course of the lifetime of a subject afflicted with a substance addiction often predicates life-long administration of therapy, be it pharmacological, behavioral, or both.

One important measure of the efficacy of a therapy for addiction is the level of desire for ingesting the substance, otherwise known as craving. Most addictive substances are clearly associated with the condition of craving following a brief period of abstinence, ranging from minutes to days, depending upon the substance and the individual. For many afflicted individuals, the phenomenon of craving may be lifelong, thus leading to the need for prolonged administration of therapy for addiction.

While craving is considered to be a psychological or strong mental desire which is difficult to resist, other aspects of substance addiction which should be targeted by specific therapies are other acute and chronic withdrawal symptoms in addition to craving. In the case of cocaine addiction, the most prominent withdrawal symptoms are subjective sensations of mild to severe dysphora, depression, anxiety or irritability. This is also true of amphetamine, alcohol, opiate and nicotine withdrawal. Thus, in addition to reducing intake or craving, another measure of the efficacy of anti-addiction therapy is to diminish the undesirable psychological effects associated with discontinuation or withdrawal from the addictive substance or product.

Addictions are associated with specific symptoms of withdrawal, depending upon the substance for which addiction had developed. For example, cigarette smokers who reduce or cease tobacco product consumption frequently experience increased appetite for food and therefore are at risk for weight gain. In fact many smokers report that the experience or fear of weight gain significantly contributes to their inability to reduce or cease their level of tobacco product consumption. Cocaine, amphetamine, and opiate habits are also initiated and promulgated for their weight loss potential. Thus the invention provides composition and therapeutic methods for the control of body weight in a subject addicted to a chemical substance or product thereof, including nicotine, amphetamines, cocaine, and opiates.

In addition, the well-known phenomena of compulsive consumption of sweets, although not formally classified as a substance addiction disorder by all medical disciplines, has many aspects in common with other addictive disorders. Thus, subjects afflicted with an addiction to frequent episodes of craving for food, in particular sweets, may benefit from the anti-craving effects of the invention.

Other well-known withdrawal symptoms associated with substance addiction are headache, sleep disturbances, including hypersomnia and insomnia, and signs of autonomic arousal such as sweating, tachycardia, diarrhea, stomach cramps, nausea and vomiting.

Nicotine addiction, as exemplified by chronic cigarette smoking, may be taken to be a valid model for other conditions of substance addiction. Many substance addictions share certain aspects in common, as delineated above with respect to craving and weight gain as withdrawal phenomena. Thus the compositions and methods for treatment of addiction disclosed herein may be considered useful for the treatment of substance addiction in general, particularly since they are believed to share a common physiological mechanism attributed to the dopaminergic, and other neurotransmitter, pathways of reward in the brain.

Inhibitors of the intracellular enzyme monoamine oxidase have been suggested to be of value in the treatment of addiction, although to date this contention has never been rigorously established by way of collection and analysis of clinical data. An inhibitor of monoamine oxidase is a compound which either inhibits in vitro the reaction catalyzed by the enzyme, or a compound which binds to the enzyme as demonstrated by in vitro or in vivo binding analysis. For purposes of the present invention, a monoamine oxidase inhibitor may be either a reversible or an irreversible inhibitor of the enzyme, Similarly, a monoamine oxidase inhibitor may be either a selective or a non-selective inhibitor with respect to the two known isoforms of the enzyme, monoamine oxidase A and monoamine oxidase B. Examples of monoamine oxidase inhibitors include, but not only, the following: selegiline (deprenyl, I-deprenyl) (R)-(−)-N,2-dimethyl-N-2-propynylphenethylamine; desmethylselegiline; S(+)desmethylselegiline, lazabemide; broformine; rasagiline; LU-53439; LU-43839; idasoxan; clorgyline (N-[3-(2′,4′-dichlorophenoxy)-N-methylpropargylamine) (May & Baker); cimoxatone, cirazoline (MD780515, MD-770222) (Synthelabo); befloxatone (MD-370503) (Synthelabo); brofaromine (Consonar, CGP-11305A) (Ciba-Geigy); bazinaprine (SR 95191) (3-(2-morpholinoethylamino)-4-cyano-6-phenylpyridazine) (Sanofi); MD-240928 (R-3-[4-((3-chlorophenyl) methoxy)phenyl]-5-[(methylamino)methyl]-2-oxazolidinone methanesulfonate (Lilly); BW-616U (Burroughs Wellcome); BW-1370U87 (1-ethylphenoxathiin-10,10-dioxide (Burroughs Wellcome); CS-722 (RS-722) ((R)4-chloro-2-(2-hydroxy-3-morpholinopropyl)-5-phenyl-4-isoxazolin-3-one hydrochloride (Sankyo); E-2011 ((5R)-3-[2-((1S)-3-cyano-1-hydroxypropyl)-benzothiazol-6-yl]-5-methoxymethyl-2-oxazolindinone (Eisai); harmine (3-phenylquinoline,3-(4-hydroxyphenyl)quinoline; harmane (1-methyl-9H-pyridol/3,4-b/indole); norharmane; harmaline; moclobemide (Aurorix, RO 11-1163) (Roche); minaprine (3-(2-morpholino-ethylamino)-4-methyl-6-phenylpyridazine; PharmaProjects 3975 (Hoechst); RO 41-1049 (N-(2-aminoethyl)-5-(3-flurophenyl)-4-thiazolecarboxemide) (Roche); RS-8359 (+/−)-4-(4-cyanoanilino)-5,6-dihydro-7-hydroxy-7H-cyclopenta[d]-pyrimidine) (Sankyo); T-794 ([(5R)-3-(6-(cyclcopropylmethoxy)2-napthealenyl)-5-(methoxymethyl)2-oxazolidone) (Tanabe Seiyaku); toloxatone (Humoryl, Perenum, MD-690276, MD-69276) (Synthelabo); K-Y 1349 (Kalir and Youdim); LY-51641 (N-[2-(o-chlorophenylxoy)ethyl]cyclopropylamine hydrochloride) (Lilly); LY-121768 (N-[2-o-iodophenoxy)ethyl]cyclopropylamine hydrochloride) (Lilly); M&B 9303 (May & Baker); MDL 72394 (Marion Merrell); MDL 72392 (Marion Merrell); sercloremine (CGP-4718A) (Ciba-Geigy); MD 72394 ((E)-beta-fluoroethylene-m-tyrosine); MO 1671; amiflamine ([S-(+)4dimethylamino-alpha,2-dimethylphenetrhylamine] (FLA-336, FLA-668, FLA-788) (Astra); vanoxerine (boxeprazine) (NovoNordisk); AGN 2253 (Nicholas Kiwi); iproniazid (Marsilid); isocarboxazid (Marplan) (Roche); M-3-PPC (Draxis); nialamid (Niamid); phenelzine (Nardil) (Parke-Davis); pargyline (Euronyl) (Abbott); tranylcypromine (Pamate) (Smith-Kline Beecham); budipine (BY-701) (t-butyl-4,4 diphenyl piperidine) (Byk Gulden); caroxazone (Timosteni, FI-6654) (Pharmacia Upjohn, Farmitalia); D-1711 (Biocodex); fezolamine (WIN-41528-2) ([N,N-dimethyl-3,4diphenyl-1H-pyrazole-1-propanamine-(E)-2-butenedioate]) (Sanofi); FLA-334 (RAN-113) (p-amino-amphetamine) (Astra); FLA-289 (FLA-299, FLA-365, FLA-384, FLA-463, FLA-727) (p-dimethylamino-amphetamine) (Astra); K-11566 (Pharmacia Upjohn, Farmitalia); K-11829 (Pharmacia Upjohn, Farmitalia); metralindole; MPCPAM (Yissum); PharmaProjects 227 (Syntex/Roche); PharmaProjects 2806 (Fournier); PharmaProjects 1122; PharmaProjects 3311 (Roche); PharmaProjects 4433 (Roche); RS-2232 (Sankyo); UP-614-04 (Bristol-Myers), hypricum perforatum, or mixtures thereof.

It is understood that, although the terms, “inhibitor of monoamine oxidase” and “addictive substance, or derivative thereof” are singular, more than one inhibitor of monoamine oxidase and more than one addictive substance, or derivative thereof, can be used concurrently in the methods of the invention.

Inhibitors of monoamine oxidase, are generally employed in the medical arts as anti-depressants and as anti-parkinson agents, but when used in combination with administration of an addictive substance, or derivative thereof, for treatment of addiction the use is a new one. Representative diseases, disorders and conditions in which inhibitors of monoamine oxidase are considered to have a therapeutic effect include: Parkinson's disease (either idiopathic or familial), parkinsonism, depression (including depression refractory to other tricyclic antidepressants, selective serotonin reuptake inhibitors), Alzheimer's disease, Huntington's disease, dementia, neurodegenerative diseases, attention deficit disorder, migraine, narcolepsy, psychiatric disorders, panic disorders, social phobias, anxiety, psychoses, obsessive-compulsive disorders, obesity or eating disorders, body dysmorphic disorders, post-traumatic stress disorders, or conditions associated with aggression. The inhibitor of monoamine oxidase is administered not only to treat symptoms of these diseases, disorders and conditions, but also to delay of prevent or delay onset or worsening of symptoms of these diseases, disorders and conditions. For example, it has been suggested that monoamine oxidase inhibitors can serve as neuroprotective agents in Parkinson's disease.

Because Parkinson's disease is caused by premature death of nerve cells in the substantia nigra of the brain, use of monoamine oxidase inhibitors can reduce or eliminate premature cell death, and thereby prevent or slow down the onset of symptoms of disease. Neuroprotection can thus be significantly valuable in the treatment of Parkinson's disease, as well as other neurodegenerative diseases which are caused by the death or dysfunction of cells in the central nervous system. Nicotine has also been implicated as a neuroprotective agent in Parkinson's disease.

Although inhibitors of monoamine oxidase, in particular selegiline, are reported as being effective in treating the foregoing conditions, neither the precise number or nature of its mechanism or mechanisms of action are known. However, there is evidence that selegiline provides neuroprotection or neuronal rescue, possibly by reducing oxidative neuronal damage, increasing the amount of the enzyme superoxide dismutase, and/or reducing dopamine catabolism. For example, selegiline has been reported to act by directly maintaining, preventing loss of, and/or assisting in, the nerve function of animals. Thus, the compositions and modes of therapy disclosed herein may be useful for the treatment of a subject suffering from one or more of the foregoing diseases, in addition to the utility as agents and therapies for the treatment of addiction.

The present invention is directed to the novel combination of a monoamine oxidase inhibitor administered together with an addictive substance, or non-addictive derivative thereof. This combination has striking therapeutic effects and, accordingly, is unexpectedly useful in treating addiction and related conditions. The clinical results disclosed herein demonstrate that administration of selegiline is indeed an effective therapy for obtaining a reduction in unwanted addictions and furthermore, that the combination of selegiline and nictoine administration is considerably more effective than administration of either two agents alone. Thus, the combination of a monoamine oxidase inhibitor with an addictive substance, or a non-addictive derivative thereof, is a highly effective mode of therapy to treat addiction. Although demonstrated herein for selegiline and nicotine, other combinations incorporating members of the same classes of compounds may be expected to be equally effective. Thus, the combination of selegiline and nicotine may be expected to reduce the level of addiction to other substances, including opiates, cocaine, amphetamines, or sweets. Alternatively, by way of example, the combination of selegiline with an amphetamine may be similarly effective in reducing the number of cigarettes smoked by an addicted smoker. It is well known that among the class of addictive substances, many compounds manifest cross-tolerance with one another, demonstrating that these compounds may behave as pharmacological analogues of one another. For treatment of any particular addictive disorder in any particular individual, the choice of which monoamine oxidase inhibitor combined with which addictive substance, or derivative thereof, will be determined by the nature of the disorder, the individual tolerance of the individual, and the knowledge and experience of the practitioner.

The effective amount can be administered in a series of doses separated by appropriate intervals, such as hours, days, or weeks. Alternatively, the effective amount can be administered as a sustained release dose, such as by a controlled-release dosage formulation.

The inhibitor of monoamine oxidase inhibitor is administered in an effective amount, which is that amount necessary to alleviate, reduce, attenuate, or eliminate the symptoms associated with the addictive disorder, disorder or condition to be treated or to slow disease progression. The monoamine oxidase inhibitor can also be administered prophylactically, in order to prevent symptoms associated with the addicitve disorder or disease, or to delay onset of symptoms associated withdrawal or abstinence. If more than one inhibitor of monoamine oxidase inhibitor is used, the effective amount is that amount of the combination of inhibitor of monoamine oxidase inhibitors that is necessary to alleviate, reduce, eliminate, prevent, or delay onset of the symptoms associated with withdrawal or abstinence. The effective amount will be determined on an individual basis, and will be based in part, on consideration of the particular monoamine oxidase inhibitor, the individual's size and gender, the severity of the symptoms to be treated, the result sought, and the disease, disorder or condition to be treated or prevented. The effective amount will generally be an amount which decreases brain monoamine oxidase activity by at least about 10%, preferably at least about 30%, more preferably at least about 50%, more preferably at least 70%, and more preferably up to 100%. Usually, the effective amount will be between approximately 0.001 mg/kg to 1 g/kg, preferably about 0.01 mg/kg to 10 mg/kg. For example, it was found that if the inhibitor of monoamine oxidase inhibitor is clorgyline, the preferred effective amount in mice to inhibit 90% of striatal inhibitor of monoamine oxidase while maintaining 50% of intestinal inhibitor of monoamine oxidase activity is 0.6 mg/kg. One of ordinary skill in the art is able to extrapolate the preferred effective amount in a subject. The effective amount can be determined by one of ordinary skill in the art, employing such factors and using no more than routine experimentation.

The effective amount can be administered in a series of doses separated by appropriate intervals, such as hours, days, or weeks. Alternatively, the effective amount can be administered as a sustained release dose, such as by a controlled-release dosage formulation.

The monoamine oxidase inhibitor, or inhibitors, is administered nasally, via a respiratory route or an intrapulmonary route, via an oral or enteral route, or via a parenteral route, i.e., by any means other than through the gastrointestinal tract or lungs. For example, the MAO inhibitor can be administered subcutaneously, transdermally, intradermally, intravenously, intramuscularly, intraperitoneally, topically, rectally, vaginally, or via an implanted reservoir. In a preferred embodiment, the MAO inhibitor is administered intradermally or transdermally. The term “transdermal” includes passive transdermal and also active electrotransdermal, e.g., iontophoretic or electroosmotic, delivery. Representative intradermal, transdermal delivery and electrotransdermal and other transdermal delivery means are described in the art. A variety of compositions of inhibitors of monoamine oxidase, in particular selegiline, are disclosed in the art, including tablets, pills, capsules, powders, aerosols, suppositories, skin patches, parenterals, and oral liquids, including oil-aqueous suspensions, solutions, emulsions, and selegiline-containing sustained release (long acting) formulations and devices.

The compounds and methods of this invention can be adapted to the formulation and use of the therapeutic compositions comprising an inhibitor of monoamine oxidase and an addictive substance, or non-addictive derivative thereof, in admixture with conventional excipients, i.e. pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

Practice of the inventive method comprises administrating an to a subject in need thereof of a first compound selected from the group of monoamine oxidase inhibitors in combination with a second compound selected from the group of addictive substances, including but not only, nicotine, opiates, amphetamines, and cocaine. The first component may be either a specific or non-specific inhibitor of monoamine oxidase isozymes. When administered in different vehicles through either the same or different routes of administration the method is known herein as co-administration; both administration of the compounds via the same vehicle or co-administration through different vehicles are acceptable forms of administration when implementing the invention.

For the purpose of implementing the invention, any compound known as a inhibitor of monoamine oxidase and any compound or composition known to be an addictive substance, or derivative thereof, are acceptable.

Additional compounds which have pharmacological actions which may serve to enhance the efficacy of the combination of an inhibitor of monoamine oxidase with an addictive substance may be optionally included within the compositions and methods of administration of the invention. Examples of compounds which may serve to benefit a subject attempting withdrawal or prolonged abstinence from an addictive substance are the following: an inhibitor of nicotine metabolism; a nicotine antagonist; a nicotine agonist; an inhibitor of CYP2A6 a beta-blocker; an alpha-2 adrenergic anatagonist; a muscle relaxant; a benzodiazepine; an inhibitor of nicotine metabolism; a nicotine antagonist; a peripheral nicotine anatagonist; a nicotine agonist; a dopamine agonist; a dopamine antagonist; a serotonin agonist; a serotonin antagonist; an antihistamine; a cannabinoid receptor antagonist; a cannabinoid receptor agonist; an anticholinergic agent; an inhibitor of acetylcholine esterase; a muscarinic antagonist; a muscarinic agonist; an opiate agonist; an opiate anatogonist, an amphetamine agonist; an amphetamine antagonist; a glutamate antagonist; a NMDA antagonist; an antihypertensive; an inhibitor of serotonin transport (reuptake); an inhibitor of dopamine transport (re-uptake); an inhibitior of noradrenaline transport (re-uptake); a NaSSA; an NMDA antagonist; an analgesic; a vasodilator; a calcium channel antagonist; a xanthine; an inhibitor of COMT (catechol-O-methyltransferase); a steroid; a steroid hormone; a vitamin; an inhibitor or monoamine oxidase; an anti-oxidant; an atypcial antidepressant; an atypical antipsychotic; an addictive substance; a pharmacological analogue of an addictive substance; or mixtures thereof.

The combination of an inhibitor of monoamine oxidase and an addictive substance, or derivative thereof, can also be combined where desired with other active agents, e.g., pharmacologically active compounds of potential value in any particular condition and any particular patient, for use as adjunctive agents in the treatment of disorders of substance addiction. Thus the invention provides for compositions comprising an amount of an inhibitor of monoamine oxidase, an amount of an addictive substance, a suitable diluent or carrier, wherein said monoamine oxidase inhibitor is selegiline, said carrier is a means for oral or parenteral delivery, and said composition further comprises at least one compound selected from the group consisting of: methoxsalen, apomorphine, bromocriptine, tryptans, sumatryptan, tacrine (Cognex), mirtazapine (Remerone), ropirplone, donepezil (Aricept), rivastigmine (Exelon, Novartis), buproprion, (Zyban, Wellbutrin), physostigmine, physostigmine analogues, tacrine, methacholine, edrophonium, scopolamine, atropine, an opiate, an amphetamine, thiamine, a xanthine, cotinine, mecamylamine, caffeine, propranolol, levodopa, carbidopa, amantadine, metamphetamine, methylphenidate, methylenedioxymethamphetamine, lofexadine, clonidine, yohimbine, phentolamine, moxonidine. SR 141716 (Synthelabo), testosterone, estradiol, progesterone, norethindrone, fentanyl, morphine, buprenorphine, sumatryptin, naloxone, dextramethorphan (ALGOS), cyclazocine (NIDA), lorazepam, alprazolam, nitroglycerin, buspirone, scopolamine, an essential vitamin, physostigmine, methacholine, edrophonium, quaternary tetraalkylammonium salts, 9-amino-1,2,3,4-tetrahydracridine, diisopropyl fluorophosphate, paraoxon, soman, arecoline, 4-aminopyridine, choline alfoscerate, eptastigmine, eseridine salicylate, galantamine hydrobromide, linopirine, RS-86, suronacrine maleate, scopolamine, atropine, benactyzine, oxybutynin, quinuclidinyl benzilate, 3-quinuclidinyl-xanthene-9-carboxylate, quinuclidinyl atrolactate, or mixtures thereof. However, not preferred are muscarinic antagonists that have limited or no central antimuscarinic effects, such as methoctramine.

While the examples provided herein describe use of the monoamine oxidase inhibitor in oral administration and the addictive substance in transdermal administration, the success described affords good evidence to suppose that other routes of administration, or combinations with other pharmaceutical preparations, would be at least as successful. The route of administration, e.g., topical, transdermal, parenteral, oral, or inhalant, and the dosage regimen will be determined by skilled clinicians, based on factors such as exact nature of the condition being treated, the severity of the condition, the age and general physical condition of the patient, and so on.

The inhibitor of monoamine oxidase inhibitor can be administered in dosage formulations containing conventional, non-toxic, physiologically-acceptable carriers, adjuvants, and/or vehicles. The formulation in which the inhibitor of monoamine oxidase inhibitor is administered will depend at least in part on the route by which it is administered. In implementation, more than one inhibitor of monoamine oxidase may be administered, including two or more monoamine oxidase inhibitors each of which exerting a selective effect on inhibition of one of the isozyme forms of the enzyme, i.e. monoamine oxidase A and monoamine oxidase B.

The daily dose can be administered in a single or multiple dosage regimen. Although the oral route of administration will generally be most convenient, drug may be administered by the parenteral, topical, transdermal dosage forms, including ointments, creams and patches, intraocular, buccal, sublingual, intranasal, inhalation, vaginal, rectal or other routes as well.

Oral dosage forms will most typically be used and are preferred but other dosage forms may also be employed and may permit, for example, a continuous release of relatively small amounts of the active ingredient from a single dosage unit, such as a transdermal patch, over the course of one or more days. This is particularly desirable in the treatment of chronic conditions such as Parkinson's disease, Alzheimer's; disease, and depression. Alternatively, it may be desirable in conditions such as ischemia or neural damage to administer one or more discrete doses by a more direct systemic route such as intravenously or by inhalation. In still other instances, localized administration, such as via the intraocular route, can be indicated.

Inhibitors of monoamine oxidase are useful when administered to a subject through a wide variety of routes of administration and dosage forms in combination with other drugs. For example the use of concomitant selegiline-amantadine in oral, peroral, enteral, pulmonary, rectal, nasal, vaginal, lingual, intravenous, intraarterial, intracardial, intramuscular, intraperitoneal, intracutaneous, and subcutaneous formulations has been described. In addition, a dosage form comprising an outer wall impermeable to selegiline but permeable to external fluids. This dosage form may have applicability for the oral, sublingual or buccal administration of selegiline.

In general, the doses utilized for the above described purposes will vary, but will be in an effective amount to exert the desired anti-disease effect. As used herein, the term “pharmaceutically effective amount” refers to an amount of a first compound, selected from the group on inhibitors of monoamine oxidase, and an amount of a second compound, selected from the group of addictive substances, or non-addictive derivatives thereof, which will produce the desired alleviation in symptoms or signs of substance addiction in a subject when administered as either a single composition, i.e. in the same vehicle, or when co-administered via either separate routes of administration or via separate vehicles. The doses utilized for any of the above-described purposes will generally be from 0.001 to about 1.00 milligrams per kilogram of body weight (mg/kg), administered up to 800 times per day for respiratory delivery, one to twenty times per day for comestible articles, one to six times per day for oral tablets or capsules, one to six times per day for injections, one to six times per day for transdermal patches or topical creams, one to six times per day for suppositories, or by continuous circulatory system infusion for pump delivery from an internal or external resevoir. When the compounds are present in the same vehicle, the ratio of monoamine oxidase inhibitor to addictive substance, or non-addictive derivative thereof, may range from 1:100 w/w to 100:1 w/w. Thus, for example, a transdermal patch for use in implementing the invention may comprise, for a 70 kg subject, from 0.7 to 70 mg selegiline and from 0.7 to 70 mg nicotine per patch dosage unit, applied from one to four times per day. Alternatively, by way of example, an oral dose of selegiline ranging from 0.7 to 70 mg may be co-administered to the subject together with a transdermal patch comprising from 0.7 to 70 mg of nicotine per patch dose unit, each of which dose may be administered at a rate of 1 to 6 times per day, in accordance with the needs and preferences of the subject or his/her practitioner.

As used herein, the term “pharmaceutically acceptable carrier” refers to formulation which is safe, and provides the appropriate delivery for the desired route of administration of an effective amount of at least one compound of the present invention. As such, all of the above-described formulations of the present invention are hereby referred to as “pharmaceutically acceptable carriers.” This term refers to as well the use of buffered formulations wherein the pH is maintained at a particular desired value, ranging from pH 3.0 to pH 10.0, in accordance with the stability of the compounds and route of administration.

For parenteral application, particularly suitable are injectable, sterile solutions, preferably oily or aqueous solutions, as well as suspensions, emulsions, or implants, including suppositories. Ampoules are convenient unit dosages.

For application by inhalation, for delivery to the respiratory epithileum, in particular the lower respiratory tract and lungs, solutions or suspensions of the compounds mixed and aerosolized or nebulized in the presence of the appropriate carrier are suitable. Thus the carrier may be a vapor, a nebulized vapor, a vapor containing particulate material, or a smoke.

For topical application, particularly for the treatment of contact dermatitis, admixture of the compounds with conventional creams or delayed release patches is acceptable.

For enteral application, particularly suitable are tablets, dragees, liquids, drops, syrup, elixir, suppositories, capsules, or comestible articles, such as a gum for chewing. Taste, sweetners, or odor agents may be added as desired. When indicated, suppositories or enema formulations may be the recommended route of administration.

Sustained or direct release compositions can be formulated, e.g., oils, liposomes, or those wherein the active compound is protected with differentially degradable coatings, e.g., by microencapsulation, multiple coatings, etc. It is also possible to freeze-dry the new compounds and use the lyophilisates obtained, for example, for the preparation of products for injection.

It will be appreciated that the actual preferred amounts of active compound in a specific case will vary according to the specific compound being utilized, the particular compositions formulated, the mode of application, and the particular situs and organism being treated. Dosages for a given host can be determined using conventional considerations, e.g., by customary comparison of the differential activities of the subject compounds and of a known agent, e.g., by means of an appropriate, conventional pharmacological protocol.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

Pharmaceutical compositions containing an inhibitor of monoamine oxidase and an addictive substance or non-addictive derivative thereof, can be prepared according to conventional techniques. For example, preparations for parenteral routes of administration of the combination, e.g., intramuscular, intradermal, subcutaneous, intravenous and intraarterial routes, can employ sterile isotonic saline solutions. Sterile isotonic buffered solutions can also be employed for intraocular administration.

Transdermal dosage unit forms of inhibitors of monoamine oxidase inhibitors can be prepared utilizing a variety of previously techniques known in the art. For example, a monolithic patch structure can be utilized in which the compound is directly incorporated into the adhesive and this mixture is cast onto a backing sheet. Alternatively, the drug can be incorporated as an acid addition salt into a multilayer patch which effects a conversion of the salt to the free base.

In a particularly preferred embodiment, the present invention is directed to a transdermal delivery composition comprising selegine and nicotine in a single patch for use in treating a condition in a human related to addiction to a substance and the desire to diminish or otherwise modify the intake of that substance. Transdermal patches comprising either nicotine or selegiline have been developed and one skilled in the art may readily adapt these carrier forms to a single patch comprising both compounds. Patches comprising a combination of selegiline, or another inhibitor of monoamine oxidase, and an addictive substance, or derivatives thereof, other than nicotine, are also objects of the invention, and may be readily prepared by one skilled in the pharmaceutical arts.

Subjects treatable by the present preparations and methods include both human and non-human subjects for which therapeutic effects of combination therapy with an inhibitor of monoamine oxidase and an addictive substance or derivative thereof are useful. Accordingly, the compositions and methods above provide especially useful therapies for mammals, especially humans.

For the purpose of the present invention, forms of delivery of pharmaceutical preparations of monoamine oxidase inhibitors, including oral, parenteral, topical, transdermal patch, and inhalation are suitable as an agent administered in a therapy regimen when co-administered with a preparation of nicotine.

Several different pharmaceutical preparations of nicotine are available, including transdermal patches comprising nicotine, comestibles (gum for chewing) comprising nicotine, and inhalants for mucosal and pulmonary delivery comprising nicotine. These preparations are available for the purpose of treatment of addiction to tobacco products as nicotine replacement therapy.

In addition, nicotine may be administered: by tablet form, for absorption through the gastrointestinal tract, although this route has generally not been preferred due to side effects and inferior absorption. However, for purposes of the present invention, this route of administration may also be considered a useful method of therapy when co-administered with a monoamine oxidase inhibitor.

Nicotine and at least one monoamine oxidase inhibitor, such as selegiline, may be co-administered though different routes of administration, as described herein for orally administered selegiline and transdermally administered nicotine, or, as an alternative, these compounds may be administered through the same route of administration for achieving the object of the invention which is to aid and facilitate for a subject the process of withdrawal from an addictive substance, in particular nicotine containing tobacco products. Thus, both selegiline and nicotine may be co-administered to a subject via transdermal route employing the use for this purpose of transdermal patches containing selegiline and nicotine to achieve the same results as described herein for co-administration of these compounds in the treatment of a subject in need thereof.

The present invention also provides for pharmaceutical compositions comprising both nicotine and at least one monoamine oxidase inhibitor. Thus, for purposes of implementing the invention, a pharmaceutical composition comprising nicotine and at least one monoamine oxidase inhibitor, such as selegiline, may be administered to a subject addicted to a substance, such as nicotine, or a nicotine-containing tobacco product, such as cigarettes, for the purpose of facilitating a desired reduction in the level of nicotine or tobacco product consumption.

A single pharmaceutical composition comprising nicotine, or a pharmacologically active derivative thereof, and a monoamine oxidase inhibitor, has distinct advantages to that of administering these therapeutic agents through different routes or through the same route of administration but as separate compositions. A pharmaceutical composition comprising both types of agents has the advantage of simplification of the treatment regimen with respect to the subject, who would therefore require the administration of a single tablet or patch comprising both nicotine and an effective monoamine oxidase inhibitor, such as selegiline, instead of two separate tablets or separate patches, for example. Combination of nicotine, or an pharmacologically equivalent derivative thereof, and an effective monoamine oxidase inhibitor as a single pharmaceutical composition affords an improved means for implementing the treatment of a subject in need of administration of both types of compounds for the purpose of treating addiction, since it is well known that administration of a single pharmaceutical composition is often better tolerated, enhances subject compliance, and prevents subject confusion when compared to the co-administration of two different compositions.

Thus the invention provides for a pharmaceutical composition comprising nicotine, or a pharmacologically equivalent derivative or salt thereof, and an effective monoamine oxidase inhibitor, together with a pharmaceutically suitable diluent or carrier.

A preferred embodiment of the invention is a pharmaceutical composition comprising nicotine, or a pharmacologically equivalent derivative or salt thereof, and an effective monoamine oxidase inhibitor, together with a pharmaceutically suitable diluent or carrier, wherein the monoamine oxidase inhibitor is selegiline and the carrier is a transdermal patch. The incorporation of nicotine or equivalent derivative thereof, and selegiline, or equivalent thereof, as a single pharmaceutical composition in the form of a transdermal patch together with suitable diluents and other carriers necessary to achieve therapeutic levels of these compounds for the purpose of treating a subject addicted to a substance, in particular, tobacco products, may be readily carried out by one with knowledge and skill in the art.

Successful use of the compositions and methods above requires employment of an effective amount of both the inhibitor of monoamine oxidase and the addictive substance or non-addictive derivative therof. Both the monoamine oxdiase inhibitor and the addictive substance may be equipotent in achieving the desired effect, yet the combination does not necessarily require a commensurately increased dosage to obtain the same level of therapeutic response confered by either compound individually. Thus, dosages necessary to attain a selegiline-like therapeutic effect may actually be lower when the monoamine oxidase inhibitor is employed in combination with nicotine. The same appears to be true for nicotine, as the combination with selegiline appears to allow for a dose reduction of nicotine sooner than when nicotine is used alone. Thus the risk of adverse effects of either the monoamine oxidase inhibitor or of the addictive substance are minimized due to the potency of the two when administered in combination.

Co-administration of pharmaceutically active compounds, i.e. drugs, is defined herein as administration to a subject of two or more active compounds wherein each compound is administered to a subject within a time proximity such that the subject is exposed to the effects of the drugs at the same time. Thus co-administration may be implemented by administering pharmaceutical compositions of two different drugs either simultaneously, or each drug preparation at different hours of the same day or week, provided that the subject is exposed to the effects of each compound at the same time. The effects of a pharmaceutically active compound on a subject may be measured either by subjective report, i.e. the subject reports a decreased craving for nicotine, or through measurement of tissue levels of the administered substance or metabolites thereof or through other means, such as measuring the level of inhibition of the activity of the enzyme monoamine oxidase or measuring the degree of occupancy of binding sites in the brain through the use of isotopically labeled monoamine oxidase compounds or derivatives thereof.

In preferred embodiment, the invention entails at least once daily administration of a monoamine oxidase inhibitor, although alternate-dosing regimens may produce the same results in terms of subjective effects or measurable tissue levels. The rate of dosing of the monoamine oxidase inhibitor is also a function of the specific active compound employed. Thus, by way of example, meclobomide is typically administered twice a day while selegiline or phenelzine are typically administered once a day.

In broader use of the inventive composition, persons attempting to abstain from nicotine intake will probably self-medicate. A recommended dose for self-medication is likely a maximum of about six capsules per day, each comprising an inhibitor of monoamine oxidase, an addicitive substance or a derivative thereof, and a physiologically acceptable diluent or carrier, administered at the rate of about one capsule per hour to one per three hours. Alternatively, as one embodiment of the invention, a subject may partake of a nicotine-containing product such as a dispenser of nicotine in vapor, smoke, aerosol, particulate aerosol, or nebulized form, in a desired manner in accordance with the subjects's needs for self-medication while, at the same time, taking from one to six daily doses of a monoamine oxidase inhibitor.

The amount of monoamine oxidase inhibitor to be administered for treating a subject addicted to a substance or product, such as a tobacco product, is a function of several factors and may be modified in accordance with one or more of these factors to achieve the desired result. Among the factors which determine the amount of monoamine oxidase inhibitor to be administered are the specific type of monoamine oxidase inhibitor to administered, the traditional clinical or manufacturer recommended dose, the individual tolerance of the patient, the co-administration of other medications, and the extent of therapeutic effect achieved at any particular dose. Thus, a typical starting dose may be decreased due to patient intolerance, such as experiencing a side effect of the medication, or a typical starting dose may be increase due to lack of desired response.

The therapeutic response due to treatment by co-administration of a monoamine oxidase inhibitor with nicotine, according to the present invention, may be either a subjective report or objective measure of the level of consumption of the addictive substance. Thus, when used for treating a subject addicted to tobacco to product, the response may be assessed by the subject's report of a decrease in the consumption of the tobacco product or a reduced sense of craving for the tobacco product, or both.

Another type of therapeutic response provided by implementation of the invention, which may be in addition to a reduction in crabing, is the subject's experience of a reduction in other discomforting withdrawal signs or symptoms experienced by the subject who is in the process of attempting to discontinue, attenuate, or curtail his or her level of addictive substance consumption. Such a withdrawal state may be characterized by anxiety, irritability, poor concentration, restlessness, impatience, depressed mood, insomnia or hypersomnia, enhanced thirst, enhanced appetite, gain of body weight, sexual dysfunction, or craving or consumption of another substance or product, in particular a substance or product known to have addictive potential for the subject. It is known that, in subjects addicted to substances such as ethanol, cocaine, amphetamines, or opiates, craving for these addictive substances may be partially releaved by self-administration of a nicotine-containing tobacco product, in particular cigarette smoking.

A preferred mode of the invention is for the treatment of a subject who is addicted to tobacco product, such as cigarette, cigar, or pipe smoking. The type of therapeutic response provided by implementation of the invention, and which may be in addition to a reduction in craving, is the subject's experience of a reduction in other discomforting withdrawal signs or symptoms experienced by the subject who is in the process of attempting to discontinue, attenuate, or curtail his or her level of tobacco product consumption. Thus, in the case of tobacco product addiction, the symptoms of nicotine withdrawal may be characterized by anxiety, irritability, poor concentration, restlessness, impatience, depressed mood, insomnia or hypersomnia, enhanced thirst, enhanced appetite, gain of body weight, sexual dysfunction, craving for the tobacco product to which the subject is addicted, craving or consumption of another tobacco product, craving or consumption of another product comprising nicotine, or craving or consumption of another substance, in particular a substance known to have addictive potential for the subject.

A significant advantage of the present invention is the co-administration of a monoamine oxidase inhibitor, such as selegiline, and nicotine replacement therapy, such as a transdermal nicotine patch, for treating a subject addicted to tobacco products and in need of specific therapy to reduce the level of consumption thereof, including achieving a state of complete abstinence. Moreover, while both monoamine oxidase inhibitors, as demonstrated herein for selegiline, and nicotine replacement therapies, such as administration of a transdermal patch, are effective modalities for a treating a subject addicted to tobacco products, the administration to a subject of the two types of drug together, i.e. co-administration of selegiline and nicotine, affords therapeutic results which surpass the efficacy of either form of therapy when administered alone. Thus the co-administration of a monoamine oxidase inhibitor and a nicotine replacement regiment, as disclosed below for co-administration to subjects in need thereof of selegiline and nicotine transdermal patch, comprises a synergistic combination previously unanticipated in the art.

Herein it is disclosed that administration of the monoamine oxidase inhibitor selegiline, which is specific in low doses for the monoamine oxidase-B isoform of the enzyme, is comparable to nicotine replacement therapy for the treatment of tobacco product addiction. Of paramount significance is the finding, as disclosed herein, that when administered as combination therapy, selegiline and nicotine are synergistic in the ability to treat the affliction of tobacco product addiction, the combination achieving greater reductions in cigarette consumption than would be expected from the sum of each form of therapy administered individually.

Pharmaceutically acceptable acid addition salts for use in the compositions of the invention are those salts which retain the biological effectiveness and properties of the free bases and which are :not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine are also suitable for forming the compositions of the invention.

Compositions for use in the inventive method for relieving craving may also be formed as tablets or capsules suitable for oral administration and have the first and second components admixed therein. The quantities of first and second components so admixed are wherein a dose comprised by one or more tablets, is effective substantially to relieve craving for a duration of time, preferably several hours, most preferably about three hours or more.

Tablets, capsules, and comestible articles of the invention optionally include one or more pharmacologically suitable component or components such as buffering agent, preservative (e.g. antioxidants), excipients and/or tabletting agents, flavoring agents, and the like. Suitable such components are well-known to the tabletting art.

Composition embodiments of the invention were formulated and administered to patients by a treating physician as will now be described for purposes of illustrating the invention.

In general, the compounds of the invention will be administered to the warm blooded mammal as pharmaceutical compositions comprising a pharmaceutically acceptable diluent or carrier. The pharmaceutical compositions may be prepared by known procedures using well-known and readily available ingredients. In making the compositions, the active ingredient will usually be mixed with a carrier, or diluted by a carrier, or enclosed within a carrier, and may be in the form of a capsule, sachet, paper, or other container. When the carrier serves as a diluent, it may be a solid, semi-solid, or liquid material which acts as a vehicle, excipient, or medium for the active ingredient. The compositions can be in the form of tablets, pills, powders, lozenges, sachets, cachets, capsules, soft or hard gleatin capsules, sterile injectable solutions, comestibles, elixirs, suspensions, emulsions, solutions, syrups, aerosols, vapors, smokes, or ointments containing, for example, up to 30% by weight of active compound, and packaged accordingly, including sterile packaged powders.

Some examples of suitable carriers, excipients, and diluents include lactose, dextrose, sucrose, sorbitol, mannitol, starches, gum, acacia, calcium phosphate, alginates, fragacanth, gelatin, calcium silicate, micro-crystalline cellulose, polyvinylpyrrolidone, cellulose, water syrup, methyl cellulose, methyl and propyl hydroxy-benzoates, talc, magnesium stearate, and mineral oil. The formulations can additionally include lubricating agents, wetting agents, emulsifying and suspending agents, preserving agents, sweetening agents, or flavoring agents. Compositions may be formulated so as to provide quick, sustained, or delayed release of the active ingredient after administration to the patient by employing procedures well known in the art.

The following formulation example is illustrative only and is not intended to limit the scope of the invention in any way.

### CLINICAL EXAMPLES

To demonstrate the therapeutic effect of selegiline and of a combination of selegiline and nicotine in the treatment of a subject addicted to tobacco products, the results of two experimental clinical trials are disclosed herein for the first time.

Experiment One

An experimental clinical trial was performed to assess the effect of selegiline alone and in combination with co-administration of nicotine. Neither the use of selegiline alone nor in combination with nicotine has been previously tested or implemented for the treatment of substance addiction and is disclosed herein for the first time.

TABLE 1
Initial Characteristics of the Trial Subjects

| | | | Average | | | Beck |
|---|---|---|---|---|---|---|
| Patient | | | Consumption | Pack | Tolerance | Depression |
| No. | Age | Sex | (cigarettes/day) | Years | Score† | Inventory‡ |
| 1 | 40 | M | 20 | 20 | 7 | 2 |
| 2 | 39 | F | 25 | 25 | 6 | 7 |
| 3 | 30 | M | 15 | 10 | 4 | 9 |
| 4 | 55 | F | 20 | 40 | 6 | 2 |
| 5 | 39 | M | 20 | 22 | 5 | 20 |
| 6 | 55 | F | 30 | 50 | 7 | 0 |
| 7 | 37 | M | 20 | 20 | 8 | 6 |
| 8 | 35 | M | 20 | 16 | 5 | 0 |
| 9 | 39 | M | 25 | 30 | 7 | 3 |

†Fagerstrom Tolerance Score (0-11); ‡Beck Depression Inventory (0-63)

In this prospective open trial subjects addicted to chronic cigarette smoking were administered either oral selegiline tablets (Jumex, Janssen, Belgium) 5 mg. b.i.d. or administered, for subjects 8 and 9, combined co-administration of both oral selegiline and nicotine replacement therapy via transdermal patch. Subjects were then monitored for their level of tobacco product consumption during the course of the next 8 weeks. The study protocol relied on the subjective report of the patient for measurement of the therapy outcome, i.e. reporting of level of cigarette consumption was elicited from the participating subjects at weekly intervals intervals throughout the course of the study, unless the patient was using nictotine replacement therapy. Initial, trial entry levels of craving were determined by administration of the Fagerström Tolerance Score exam, while initial level of mood was monitored by administering the Beck Depression Inventory. These initial characteristics of the patient characteristics are summarized in Table 1.

Following one week on the medication, the subjects were instructed to quit smoking on “quit day”, as predetermined at the initiation of the study. As shown in Table 2, below, three subjects taking selegiline alone successfully abstained from cigarette smoking while three other patients did not respond and eventually left the study. One subject significantly reduced the number of

TABLE 2
Selegiline Alone and In Combination with Nicotine

| Sub- | | | | | | | | | | | | |
|---|---|---|---|---|---|---|---|---|---|---|---|---|
| ject | | Weeks from Quit Day | | | | | | | | | | |
| No. | | −2 | 0 | 1 | 2 | 3 | 4 | 5 | 6 | 7 | 8 | 9 |
| 1 | Cig/wk | 140 | | 0 | 0 | 3 | 0 | 0 | 0 | 0 | — | — |
| | med | — | | S | S | S | S | S | S | S | — | — |
| 2 | Cig/wk | 150 | | 0 | 0 | 0 | 0 | 0 | 0 | 0 | 0 | 0 |
| | med | — | | S | S | S | S | — | — | — | — | — |
| 3 | Cig/wk | 105 | | 0 | 0 | 0 | 0 | 0 | 0 | 0 | 0 | 0 |
| | med | — | | S | S | S | S | S | S | S | S | — |
| 4 | Cig/wk | 140 | | 21 | 21 | 25 | 25 | 25 | 14 | 21 | 21 | 21 |
| | med | — | | S | S | S | S | S | SN | S | S | S |
| 5 | Cig/wk | 140 | | 140 | — | — | — | — | — | — | — | — |
| | med | — | | S | — | — | — | — | — | — | — | — |
| 6 | Cig/wk | 210 | | 210 | — | — | — | — | — | — | — | — |
| | med | — | | S | — | — | — | — | — | — | — | — |
| 7 | Cig/wk | 140 | | 35 | 70 | — | — | — | — | — | — | — |
| | med | — | | S | S | — | — | — | — | — | — | — |
| 8 | Cigfwk | 140 | | 0 | 0 | 0 | 0 | 0 | 0 | 0 | 0 | — |
| | med | — | | SN | SN | SN | SN | SN | SN | SN | SN | — |
| 9 | Cig/wk | 140 | | 0 | 0 | 0 | 0 | 0 | 21 | 35 | 60 | 25 |
| | med | — | | N | N | N | N | N | N | N | N | SN |

Legend

S=selegiline; N=nicotine; SN=selegiline and nicotine; --=none

Med=medication; Cig/wk=cigarettes consumed per week; ----=trial dropout

cigarettes smoked. One subject (No. 8) initially, receiving both selegiline and nicotine replacement, had not smoked a single cigarette during the first eight weeks of the study. One subject (No. 9) receiving just nicotine patch alone resumed smoking after the fifth week of abstinence, but following the addition of oral selegiline, reduced the daily consumption of cigarettes smoked. In addition, subject No. 4 reduced her daily cigarette intake even further upon adding administration of nicotine patch at the sixth week of the quit day, Later she discontinued the patch, expressing that she wished to continue smoking her three post-meal cigarettes per day.

Surprisingly, while the efficacy of tobacco replacement therapy, when administered as sole therapy in the form of transdermal patch, is known to be about 20%, and that for selegiline as sole therapy, as demonstrated above, is about 30%, as disclosed herein in Table 2, 100% of the subjects receiving the combined therapy were treated successfully, i.e. abstaining or significantly reducing their level of cigarette smoking while undergoing co-administration of the two pharmaceutical compositions.

The results of the study presented herein demonstrate firstly that administration of selegiline is effective treatment for substance addiction, namely cigarette smoking. Moreover, the combination of selegiline and nicotine is more superior to either drug alone when used for this purpose, and addition of selegiline to nicotine replacement therapy is a synergistic use of the combination, particularly effective when used to treat smokers who wish to further attenuate their level of cigarette consumption when undergoing nicotine replacement therapy alone as an abstinence promoting therapy.

In this study, at follow-up one year from the initiation of treatment, subjects no. 2, 8 and 9 continued to demonstrate continuous abstinence from smoking, providing a success rate of 100% for the inventive combination of selegeline with nicotine patch compared to 14.2% for selegeline alone.

According to these results, the success rate of combination therapy is 100%, a figure which is much higher than that reported for either selegeline alone (14.2%, see above) or nicotine patch alone (9.8% N. Eng J Med 1999: 340:685-91), and which indicates a synergism for the inventive combination of selegeline and nicotine patch regimen.

Experiment Two

The second experiment is a randomized double-blind placebo controlled clinical trial wherein subjects received either selegiline in oral tablet form, or a placebo, initiating administration of this drug one week prior to the date set for self-induced reduction or cessation of tobacco product consumption, in this case cigarette smoking. Four hundred chronic smokers of both sexes subjects addicted to chronic cigarette smoking are planned to enter the study; 50 patients have been recruited already.

Subjects were admitted to the study after undergoing medical examination including anamnesis or other medical conditions and other forms of substance abuse and all signed informed consent, including the use of placebo in place of selegiline. All of the subjects admitted to the study were chronic cigarette smokers, consuming at least 15 cigarettes daily for the preceding year, but in otherwise general good physical and free of concomitant medication use. The study was approved and subjected to regulation by both a hospital Helsinki committee and the Israel Ministry of Health.

Subjects were assigned to one of two groups: oral selegeline tablet 5 mg (Jumex, Jansson, Belgium) twice a day for 26 weeks in combination with nicotine skin patch for 8 weeks or oral placebo twice a day for 26 weeks combined with nicotine skin patch for 8 weeks. Self-reported abstinence, urine cotinine levels, safety laboratory parameters and adverse events were monitored at regular intervals.

During the initial phase of the trial, half of the patients were administered either selegiline tablets taken as 5 mg tablet twice a day, once in the morning and once in the afternoon. Alternately, to the second half of patients, instead of selegiline, a placebo tablet was provided, identical in shape, colour, marking, weight, and size to the selegiline-containing tablets. In all cases the physician investigator supervising the trial was blinded to the identity of the active selegiline tablet vs the placebo tablet.

According to the initial results of this long term abstinence study, a high degree of success was noted for smokers treated with the combination therapy of oral selegeline and nicotine patch (first 8 weeks only) regimen. From among a group of 45 smokers who participated in the long-term clinical trial, 15 smokers demonstrated continous abstinence from smoking, as shown in Table 3 below. In this study, participants were interviewed once every two weeks for the first three months and then at six months and twelve months (where applicable) from the intiation of treatment. In addition to self-reporting, urinalysis for co-nicotine was negative on each of the four occasions conducted following conclusion of the initial eight week patch therapy.

According to these results, the success rate of combination therapy is 33%, a figure which is much higher than that reported for either selegeline alone (14.2%, see above) or nicotine patch alone (9.8%-N. Eng J Med 1999; 340:685-91), and which indicates a synergism for the inventive combination of selegeline and nicotine patch regimen.

TABLE 3
Characteristics of Quitters (Continous Abstinence from Smoking)
Treated by Oral Selegeline and Nicotine Patch Combination Therapy

| | | | | Continuous |
|---|---|---|---|---|
| Participant | | | | abstinence |
| no. | Gender | Age | Pack-years | (months) |
| 1 | F | 42 | 36 | 12 |
| 2 | M | 29 | 15 | 12 |
| 3 | F | 40 | 25 | 12 |
| 4 | F | 38 | 30 | 12 |
| 5 | M | 35 | 24 | 6 |
| 6 | F | 42 | 13 | 6 |
| 7 | M | 47 | 29 | 6 |
| 8 | M | 51 | 40 | 6 |
| 9 | M | 36 | 20 | 6 |
| 10 | M | 51 | 33 | 6 |
| 11 | M | 47 | 45 | 6 |
| 12 | M | 38 | 29 | 6 |
| 13 | F | 43 | 48 | 6 |
| 14 | M | 43 | 26 | 6 |
| 15 | F | 49 | 90 | 6 |

What we claim is:

1. A pharmaceutical composition comprising an amount of an inhibitor of monoamine oxidase, an amount of an addictive substance or derivative thereof, and a suitable diluent or carrier.

2. A composition as of claim 1, wherein said composition is adapted to administer both the inhibitor of monoamine oxidase and the addictive substance, or derivative thereof, via the same route of administration.

3. A composition as of claim 1, wherein said carrier is an oral tablet, capsule, or comestible article.

4. A composition as of claim 1, wherein said carrier is an aerosol, nebulized vapor, or smoke.

5. A composition as of claim 1, wherein said carrier is a transdermal patch.

6. A composition as of claim 1, wherein said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration.

7. A composition as of claim 1, wherein said addictive substance, or derivative thereof, is nicotine and said carrier is an oral tablet, capsule, or comestible article.

8. A composition of claim 1, wherein said addictive substance, or derivative thereof, is nicotine and said carrier is an aerosol, vapor, or smoke carrier.

9. A composition as of claim 1, wherein said addictive substance, or derivative thereof, is nicotine and said carrier is a transdermal patch.

10. A composition as of claim 1, wherein said addictive substance, or derivative thereof, is nicotine and said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration.

11. A composition of claim 1, wherein said monoamine oxidase inhibitor is selegiline and said carrier is an oral tablet, capsule, or comestible article.

12. A composition as of claim 1, wherein said monoamine oxidase inhibitor is selegiline and said carrier is an aerosol, vapor, or smoke carrier.

13. A composition of claim 1, wherein said monoamine oxidase inhibitor is selegiline and said carrier is a transdermal patch.

14. A composition as of claim 1, wherein said monoamine oxidase inhibitor is selegiline and said carrier is suitable for parenteral administration, including intradermal, subcutaneous, intramuscular, Intravenous, intrathecal, sublingual, rectal, vaginal, intraocular, transdermal, respiratory mucosal, or pulmonary routes of administration.

15. A composition as of claim 1, wherein said monoamine oxidase inhibitor is selegiline, said addictive substance, or derivative thereof, is nicotine, and said carrier is an oral tablet, capsule, or comestible article.

16. A composition as of claim 1, wherein said monoamine oxidase inhibitor is selegiline, said addictive substance, or derivative thereof, is nicotine, and said carrier is an aerosol, vapor, or smoke carrier.

17. A composition as of claim 1, wherein said monoamine oxidase inhibitor is selegiline, said addictive substance, or derivative thereof, is nicotine, and said carrier is a transdermal patch.

18. A composition as of claim 1, wherein said monoamine oxidase inhibitor is selegiline, said addictive substance, or derivative thereof, is nicotine, and said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration.

19. A composition as of claim 1, wherein said addictive substance, or derivative thereof, is nicotine, said carrier is an oral tablet, capsule, or comestible article, and said inhibitor of monoamine oxidase is selected from the group consisting of selegiline, desmethylselegiline, S(+)desmethylselegiline, lazabemide, broformine, rasagiline, LU-53439, LU-43839, idazoxan, clorgyline; cimoxatone; cirazoline; befloxatone; brofaromine; bazinaprine; MD-240928; BW-616U; BW-1370U87; CS-722; E-2011; harmine; harmane, norharmane; harmaline; moclobemide; PharmaProjects 3975; RO 41-1049; RS-8359; T-794; toloxatone; K-Y 1349; LY-51641; LY-121768; M&B 9303; MDL 72394; MDL 72392; sercloremine; MD 72394; MO 1671, amiflamine; vanoxerine; AGN 2253; iproniazid; isocarboxazid; M-3-PPC; nialamid; phenelzine; pargyline; tranylcypromine, budipine; caroxazone; D-1711; fezolamine; FLA-334; FLA-289; K-11566; K-11829; metralindole; MPCPAM; PharmaProjects 227; PharmaProjects 2806; PharmaProjects 1122; PharmaProjects 331; PharmaProjects 4433; RS-2232; UP-614-04, or mixtures thereof.

20. A composition as of claim 1, wherein said addictive substance, or derivative thereof, is nicotine, said carrier is an aerosol, vapor, or smoke carrier, and said inhibitor of monoamine oxidase is selected from the group consisting of selegiline, desmethylselegiline, S(+)desmethylselegiline, lazabemide, broformine, rasagiline LU-53439, LU-43839, idazoxan, clorgyline; cimoxatone, cirazoline; befloxatone; brofaromine; bazinaprine; MD-240928; BW616U; BW-1370U87 ; CS-722; E-2011; harmine; harmane, norharmane; harmaline; moclobemide; PharmaProjects 3975 ; RO 41-1049; RS-8359; T-794; toloxatone; K-Y 1349; LY-51641; LY-121768; M&B 9303; MDL 72394; MDL 72392; sercloremine; MD 72394; MO 1671, amiflamine; vanoxerine; AGN 2253; iproniazid; isocarboxazid; M-3-PPC; nialamid; phenelzine; pargyline; tranylcypromine, budipine; caroxazone; D-1711; fezolamine; FLA-334; FLA-289; K-11566; K-11829; metralindole; MPCPAM; PharmaProjects 227; PharmaProjects 2806; PharmaProjects 1122; PharmaProjects 331; PharmaProjects 4433; RS-2232; UP-614-04, or mixtures thereof.

21. A composition as of claim 1, wherein said addictive substance, or derivative thereof, is nicotine, said carrier is a transdermal patch, and said inhibitor of monoamine oxidase is selected from the group consisting of selegiline, desmethylselegiline, S(+)desmethylselegiline, lazabemide, broformine, rasagiline, LU-53439, LU-43839, idazoxan, clorgyline; cimoxatone, cirazoline; befloxatone; brofaromine; bazinaprine; MD-240928; BW-616U; BW-1370U87; CS-722; E-2011; harmane; harmane, norharmane; harmaline; moclobemide; PharmaProjects 3975; RO 41-1049; RS-8359; T-794; toloxatone; K-Y 1349; LY-51641; LY-121768, M&B 9303; MDL 72394; MDL 72392; sercloremine; MD 72394; MO 1671, amiflamine; vanoxerine; AGN 2253; iproniazid; isocarboxazid; M-3-PPC; nialamid; phenelzine; pargyline; tranylcypromine, budipine; caroxazone; D-1711; fezolamine; FLA-334; FLA-289; K-11566; K-11829; metralindole; MPCPAM; PharmaProjects 227; PharmaProjects 2806; PharmaProjects 1122; PharmaProjects 331; PharmaProjects 4433; RS-2232; UP-614-04, or mixtures thereof.

22. A composition as of claim 1, wherein said addictive substance, or derivative thereof, is nicotine, said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration, and said inhibitor of monoamine oxidase is selected from the group consisting of selegiline, desmethylselegiline, S(+)desmethylselegiline, lazabemide, broformine, rasagiline, LU-53439, LU-43839, idazoxan, clorgyline; cimoxatone, cirazoline; befloxatone; brofaromine; bazinaprine; MD-240928; BW-616U; BW-1370U87; CS-722; E-2011; harmine; harmane, norharmane; harmaline; moclobemide; PharmaProjects 3975; RO 41-1049; RS-8359; T-794; toloxatone; K-Y 1349; LY-51641; LY-121768; M&B 9303; MDL 72394; MDL 72392; sercloremine; MD 72394; MO 1671, amiflamine; vanoxerine; AGN 2253; iproniazid; isocarboxazid; M-3-PPC; nialamid; phenelzine; pargyline; tranylcypromine, budipine; caroxazone; D-1711; fezolamine; FLA-334; FLA-289; K-11566; K11829; metralindole MPCPAM; PharmaProjects 227; PharmaProjects 2806; PharmaProjects 1122; PharmaProjects 331; PharmaProjects 4433; RS-2232; UP-614-04, or mixtures thereof.

23. A composition as of claim 1, wherein said monoamine oxidase inhibitor is selegiline, said carrier is an oral tablet, capsule, or comestible article, and said addictive substance is nicotine, cotinine, a nicotinic agonist, a derivative of nicotine, a dopamine agonist, an opiate, morphine, heroin, methadone, buprenorphine, fentanyl, naloxone, codeine, an amphetamine, metamphetamine, methylphenidate, methylenedioxymethamphetamine, a dopamine re-uptake inhibitor, an alkaloid, cocaine, or derivative thereof.

24. A composition as of claim 1, wherein said monoamine oxidase inhibitor is selegiline, said carrier is an aerosol, vapor, or smoke carrier, and said addictive substance, or derivative thereof, is nicotine, cotinine, a nicotinic agonist, a derivative of nicotine, a dopamine agonist, an opiate, morphine, heroin, methadone, buprenorphine, fentanyl, naloxone, codeine, an amphetamine, metamphetamine, methylphenidate, methylenedioxymethamphetamine, a dopamine re-uptake inhibitor, an alkaloid, cocaine, or derivative thereof.

25. A composition as of claim 1, wherein said monoamine oxidase inhibitor is selegiline, said carrier is a transdermal patch, and said addictive substance, or derivative thereof, is nicotine, cotinine, a nicotinic agonist, a derivative of nicotine, a dopamine agonist, an opiate, morphine, heroin, methadone, buprenorphine, fentanyl, naloxone, codeine, an amphetamine, metamphetamine, methylphenidate, methylenedioxymethamphetamine, a dopamine re-uptake inhibitor, an alkaloid, cocaine, or derivative thereof.

26. A composition as of claim 1, wherein said monoamine oxidase inhibitor is selegiline, said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration, and said addictive substance, or derivative thereof, is nicotine, cotinine, a nicotinic agonist, a derivative of nicotine, a dopamine agonist, an opiate, morphine, heroin, methadone, buprenorphine, fentanyl, naloxone, codeine, an amphetamine, metamphetamine, methylphenidate, methylenedioxymethamphetamine, a dopamine re-uptake inhibitor, an alkaloid, cocaine, or derivative thereof.

27. A composition of claim 1, wherein said monoamine oxidase inhibitor is selegiline, said addictive substance, or derivative thereof, is nicotine, said carrier is an oral tablet capsule, or comestible article, and said composition further comprises at least one compound selected from the group consisting of an inhibitor of nicotine metabolism; a nicotine antagonist; a nicotine agonist; an inhibitor of CYP2A6, a beta-blocker; an alpha-2 adrenergic anatagonist; a muscle relaxant; a benzodiazepine; an inhibitor of nicotine metabolism; a nicotine antagonist; a peripheral nicotine anatagonist; a nicotine agonist; a dopamine agonist; a dopamine antagonist; a serotonin agonist; a serotonin antagonist; an antihistamine; a cannabinoid receptor antagonist; a cannabinoid receptor agonist; an anticholinergic agent; an inhibitor of acetylcholine esterase; a muscarinic antagonist; a muscarinic agonist; an opiate agonist; an opiate anatogonist; an amphetamine agonist; an amphetamine antagonist; a glutamate antagonist; a NMDA antagonist; an antihypertensive; an inhibitor of serotonin transport (re-uptake); an inhibitor of dopamine transport (re-uptake); an inhibitior of noradrenaline transport (re-uptake); a NaSSA; an NMDA antagonist; an analgesic; a vasodilator; a calcium channel antagonist; a xanthine; an inhibitor of COMT (catechol-O-methyltransferase); a steroid; a steroid hormone; a vitamin, an inhibitor or monoamine oxidase; an anti-oxidant; an atypcial antidepressant; an atypical antipsychotic; an addictive substance; a pharmacological analogue of an addictive substance; or mixtures thereof.

28. A composition as of claim 1, wherein said monoamine oxidase inhibitor is selegiline, said addictive substance, or derivative thereof, is nicotine, said carrier is an aerosol, vapor, or smoke carrier, and said composition further comprises at least one compound selected from the group consisting of: an inhibitor of nicotine metabolism; a nicotine antagonist; a nicotine agonist; an inhibitor of CYP2A6, beta-blocker; an alpha-2 adrenergic anatagonist; a muscle relaxant; a benzodiazepine; an inhibitor of nicotine metabolism; a nicotine antagonist; a peripheral nicotine anatagonist; a nicotine agonist; a dopamine agonist; a dopamine antagonist; a serotonin agonist; a serotonin antagonist; an antihistamine; a cannabinoid receptor antagonist; a cannabinoid receptor agonist; an anticholinergic agent; an inhibitor of acetylcholine esterase a muscarinic antagonist; a muscarinic agonist; an opiate agonist, an opiate anatogonist; an amphetamine agonist; an amphetamine antagonist; a glutamate antagonist; a NMDA antagonist; an antihypertensive; an inhibitor of serotonin transport (re-uptake); an inhibitor of dopamine transport (re-uptake); an inhibitior of noradrenaline transport (re-uptake); a NaSSA; an NMDA antagonist; an analgesic; a vasodilator; a calcium channel antagonist; a xanthine; an inhibitor of COMT (catechol-O-methyltransferase); a steroid; a steroid hormone; a vitamin; an inhibitor or monoamine oxidase; an anti-oxidant; an atypcial antidepressant; an atypical antipsychotic; an addictive substance: a pharmacological analogue of an addictive substance; or mixtures thereof.

29. A composition as of claim 1, wherein said monoamine oxidase inhibitor is selegiline, said addictive substance, or derivative thereof, is nicotine, said carrier is a transdermal patch, and said composition further comprises at least one compound selected from the group consisting of: an inhibitor of nicotine metabolism; a nicotine antagonist; a nicotine agonist; an inhibitor of CYP2A6, a beta-blocker; an alpha-2 adrenergic anatagonist; a muscle relaxant; a benzodiazepine; an inhibitor of nicotine metabolism; a nicotine antagonist; a peripheral nicotine antagonist; a nicotine agonist; a dopamine agonist; a dopamine antagonist; a serotonin agonist; a serotonin antagonist; an antihistamine; a cannabinoid receptor antagonist; a cannabinoid receptor agonist; an anticholinergic agent; an inhibitor of acetylcholine esterase; a muscarinic antagonist; a muscarinic agonist; an opiate agonist; an opiate anatogonist; an amphetamine agonist; an amphetamine antagonist; a glutamate antagonist; a NMDA antagonist; an antihypertensive; an inhibitor of serotonin transport (re-uptake); an inhibitor of dopamine transport (re-uptake); an inhibitior of noradrenaline transport (re-uptake); a NaSSA; an NMDA antagonist; an analgesic; a vasodilator; a calcium channel antagonist; a xanthine; an inhibitor of COMT (catechol-O-methyltransferase); a steroid; a steroid hormone; a vitamin; an inhibitor or monoamine oxidase; an anti-oxidant; an atypical antidepressant; an atypical antipsychotic; an addictive substance; a pharmacological analogue of an addictive substance; or mixtures thereof.

30. A composition as of claim 1, wherein said monoamine oxidase inhibitor is selegiline, said addictive substance, or derivative thereof, is nicotine, said carrier is suitable for parenteral administration, including intradermal, subcutaneous, intramuscular, intravenous, intrathecal, sublingual, rectal, vaginal, intraocular, transdermal, respiratory mucosal, or pulmonary routes of administration, and said composition further comprises at least one compound selected from the group consisting of: an inhibitor of nicotine metabolism; a nicotine antagonist; a nicotine agonist; an inhibitor of CYP2A6, a beta-blocker; an alpha-2 adrenergic anatagonist; a muscle relaxant; a benzodiazepine; an inhibitor of nicotine metabolism; a nicotine antagonist; a peripheral nicotine anatagonist; a nicotine agonist; a dopamine agonist; a dopamine antagonist; a serotonin agonist; a serotonin antagonist; an antihistamine; a cannabinoid receptor antagonist; a cannabinoid receptor agonist; an anticholinergic agent; an inhibitor of acetylcholine esterase; a muscarinic antagonist; a muscarinic agonist; an opiate agonist; an opiate anatogonist; an amphetamine agonist; an amphetamine antagonist; a glutamate antagonist; a NMDA antagonist; an antihypertensive; an inhibitor of serotonin transport (re-uptake); an inhibitor of dopamine transport (re-uptake); an inhibitor of noradrenaline transport (re-uptake); a NaSSA; an NMDA antagonist; an analgesic; a vasodilator; a calcium channel antagonist; a xanthine; an inhibitor of COMT (catechol-O-methyltransferase); a steroid; a steroid hormone; a vitamin; an inhibitor or monoamine oxidase; an anti-oxidant; an atypcial antidepressant; an atypical antipsychotic; an addictive substance; a pharmacological analogue of an addictive substance; or mixtures thereof.

31. A method of treating a subject addicted to an addictive substance or product comprising the steps if co-administering to the subject in need thereof a pharmaceutical composition comprising a therapeutically effective amount of selegiline and a pharmaceutical composition comprising a therapeutically effective amount of nicotine, thereby attenuating consumption of said substance or product.

32. A method of treating a subject addicted to an addictive substance or product comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in a transdermal patch dosage form, thereby attenuating consumption of said substance or product.

33. A method of treating a subject addicted to an addictive substance or product comprising the steps of co-administering to the subject in need thereof a pharmaceutical composition comprising a therapeutically effective amount of selegiline in oral tablet, capsule, or comestible article form and a pharmaceutical composition comprising a therapeutically effective amount of nicotine in oral tablet, capsule, or comestible article form, thereby attenuating consumption of said substance or product.

34. A method of treating a subject addicted to an addictive substance or product comprising the steps of co-administering to the subject in need thereof a pharmaceutical composition comprising a therapeutically effective amount of selegiline in oral tablet or comestible dosage form and a pharmaceutical composition comprising a therapeutically effective amount of nicotine in an aerosol, vapor, or smoke dosage form, thereby attenuating consumption of said substance or product.

35. A method of treating a subject addicted to an addictive substance or product comprising the steps of co-administering to the subject in need thereof a pharmaceutical composition comprising a therapeutically effective amount of selegiline in a transdermal patch dosage form and a pharmaceutical composition comprising a therapeutically effective amount of nicotine in a transdermal patch dosage form, thereby attenuating consumption of said substance or product.

36. A method of treating a subject addicted to an addictive substance or product comprising the steps of administering to the subject in need thereof a pharmaceutical composition comprising a therapeutically effective amount of selegiline and a therapeutically effective amount of nicotine combined in an oral tablet, capsule, or comestible dosage form, thereby attenuating consumption of said substance or product.

37. A method of treating a subject addicted an addictive substance or product comprising the steps of administering to the subject in need thereof a pharmaceutical composition comprising a therapeutically effective amount of selegiline and a therapeutically effective amount of nicotine combined in a transdermal patch dosage form, thereby attenuating consumption of said substance or product.

38. A method of treating the symptoms of nicotine withdrawal comprising the steps of co-administering to the subject in need thereof a pharmaceutical composition comprising a therapeutically effective amount of selegiline, combined with a suitable diluent or carrier, and a pharmaceutical composition comprising a therapeutically effective amount of nicotine, combined with a suitable diluent or carrier, thereby alleviating the symptoms of nicotine withdrawal.

39. A method of treating the symptoms of nicotine withdrawal comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in a transdermal patch dosage form, thereby alleviating the symptoms of nicotine withdrawal.

40. A method of treating the symptoms of nicotine withdrawal comprising the steps of co-administering to the subject in need thereof a pharmaceutical composition a therapeutically effective amount of selegiline in oral tablet, capsule, or comestible dosage form and a pharmaceutical composition comprising a therapeutically effective amount of nicotine in an oral tablet, capsule, or comestible dosage form, thereby alleviating the symptoms of nicotine withdrawal.

41. A method of treating the symptoms of nicotine withdrawal comprising the steps of co-administering to the subject in need thereof a pharmaceutical composition comprising a therapeutically effective amount of selegiline in oral dosage form and a pharmaceutical composition comprising a therapeutically effective amount of nicotine in an aerosol, vapor, or smoke dosage form, thereby alleviating the symptoms of nicotine withdrawal.

42. A method of treating the symptoms of nicotine withdrawal comprising the steps of co-administering to the subject in need thereof a pharmaceutical composition comprising a therapeutically effective amount of selegiline in a transdermal patch dosage form and a pharmaceutical composition comprising a therapeutically effective amount of nicotine in a transdermal patch dosage form, thereby alleviating the symptoms of nicotine withdrawal.

43. A method of treating the symptoms of nicotine withdrawal comprising the steps of administering to the subject in need thereof a pharmaceutical composition comprising a therapeutically effective amount of selegiline and a therapeutically effective amount of nicotine combined in an oral tablet, capsule, or comestible dosage form, thereby alleviating the symptoms of nicotine withdrawal.

44. A method of treating the symptoms of nicotine withdrawal comprising the steps of administering to the subject in need thereof a pharmaceutical composition comprising a therapeutically effective amount of selegiline and a therapeutically effective amount of nicotine combined in a transdermal patch dosage form, thereby alleviating the symptoms of nicotine withdrawal.

45. A method of treating a subject addicted to a tobacco product comprising the steps of co-administering to the subject in need thereof a pharmaceutical composition comprising a therapeutically effective amount of selegiline and a pharmaceutical composition comprising a therapeutically effective amount of nicotine, thereby attenuating tobacco consumption.

46. A method of treating a subject addicted to a tobacco product comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in a transdermal patch dosage form, thereby attenuating tobacco consumption.

47. A method of treating a subject addicted to a tobacco product comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in an oral tablet, capsule, or comestible dosage form, thereby attenuating tobacco consumption.

48. A method of treating a subject addicted to a tobacco product comprising the steps of co-administering to the subject in need thereof a pharmaceutical composition comprising a therapeutically effective amount of selegiline in oral tablet, capsule, or comestible dosage form and a pharmaceutical composition comprising a therapeutically effective amount of nicotine in an aerosol, vapor, or smoke dosage form, thereby attenuating tobacco consumption.

49. A method of treating a subject addicted to a tobacco product comprising the steps of co-administering to the subject in need thereof a pharmaceutical composition comprising a therapeutically effective amount of selegiline in a transdermal patch dosage form and a pharmaceutical composition comprising a therapeutically effective amount of nicotine in a transdermal patch dosage form, thereby attenuating tobacco consumption.

50. A method of treating a subject addicted to a tobacco product comprising the steps of administering to the subject in need thereof a pharmaceutical composition comprising a therapeutically effective amount of selegiline and a therapeutically effective amount of nicotine combined in an oral tablet, capsule, or comestible dosage form, thereby attenuating tobacco consumption.

51. A method of treating a subject addicted to a tobacco product comprising the steps of administering to the subject in need thereof a pharmaceutical composition comprising a therapeutically effective amount of selegiline and a therapeutically effective amount of nicotine combined in a transdermal patch dosage form, thereby attenuating tobacco consumption.

52. A method of treating a subject addicted to an addictive substance or product comprising the steps of administering to the subject in need thereof a pharmaceutical composition of claims 1 or 2, thereby attenuating chronic craving symptoms of a subject addicted to said substance or product.

53. A method of treating a subject addicted to an addictive substance or product comprising the steps of administering to the subject in need thereof a pharmaceutical composition of claims 3, 7, 11, 15, 19, 23, or 27, thereby attenuating chronic craving symptoms of a subject addicted to said substance or product.

54. A method of treating a subject addicted to an addictive substance or product comprising the steps of administering to the subject in need thereof a pharmaceutical composition of claims 4, 8, 12, 16, 20, 24, or 28, thereby attenuating chronic craving symptoms of a subject addicted to substance or product.

55. A method of treating a subject addicted to an addictive substance or product comprising the steps of administering to the subject in need thereof a pharmaceutical composition of claims 5, 9, 13, 17, 21, 25, or 29, thereby attenuating chronic craving symptoms of a subject addicted to said substance or product.

56. A method of treating a subject addicted to an addictive substance or product comprising the steps of administering to the subject in need thereof a pharmaceutical composition of claims 6, 10, 14. 18, 22, 26, or 30, thereby attenuating chronic craving symptoms of a subject addicted to said substance or product.

57. A method of treating the symptoms of nicotine withdrawal comprising the steps of administering to the subject in need thereof a pharmaceutical composition of claims 1 or 2, thereby alleviating the chronic craving symptoms of a subject addicted to nicotine.

58. A method of treating the symptoms of nicotine withdrawal comprising the steps of administering to the subject in need thereof a pharmaceutical composition of claims 3, 7, 11, 15, 19, 23, or 27, thereby alleviating the chronic craving symptoms of a subject addicted to nicotine.

59. A method of treating the symptoms of nicotine withdrawal comprising the steps of administering to the subject in need thereof a pharmaceutical composition of claims 4, 8, 12, 16, 20, 24, or 28, thereby alleviating the chronic craving symptoms of a subject addicted to nicotine.

60. A method of treating the symptoms of nicotine withdrawal comprising the steps of administering to the subject in need thereof a pharmaceutical composition of claims 5, 9, 13, 17, 21, 25, or 29, thereby alleviating the chronic craving symptoms of a subject addicted to nicotine.

61. A method of treating the symptoms of nicotine withdrawal comprising the steps of administering to the subject in need thereof a pharmaceutical composition of claims 6, 10, 14, 18, 22, 26, or 30, thereby alleviating the chronic craving symptoms of a subject addicted to nicotine.

62. A method of treating a subject addicted to tobacco product consumption comprising the steps of administering to the subject in need thereof a pharmaceutical composition of claims 1 or 2, thereby attenuating chronic tobacco consumption.

63. A method of treating a subject addicted to tobacco product consumption comprising the steps of administering to the subject in need thereof a pharmaceutical composition of claims 3, 7, 11, 15, 19, 23, or 27, thereby attenuating chronic tobacco consumption.

64. A method of treating a subject addicted to tobacco product consumption comprising the steps of administering to the subject in need thereof a pharmaceutical composition of claims 4, 8, 12, 16, 20, 24, or 28, thereby attenuating chronic tobacco consumption.

65. A method of treating a subject addicted to tobacco product consumption comprising the steps of administering to the subject in need thereof a pharmaceutical composition of claims 5, 9, 13, 17, 21, 25, or 29, thereby attenuating chronic tobacco consumption.

66. A method of treating a subject addicted to tobacco product consumption comprising the steps of administering to the subject in need thereof a pharmaceutical composition of claims 6, 10, 14, 18, 22, 26, or 30, thereby attenuating chronic tobacco consumption.
